(19) United States
(12) Patent Application Publication
(10) Pub. No.: US 2025/0082446 A1
(43) Pub. Date: Mar. 13, 2025

(54) Systems and Methods for Orthodontic Imaging based on Optical Coherence Tomography (OCT) Scanning and Cone Beam Computed Tomography (CBCT)

(71) Applicant: Perceptive Technologies, Inc., Boston, MA (US)

(72) Inventors: Christopher John Ciriello, Boston, MA (US); Angela Zhang, Stow, MA (US); Phillip Getto, Wellesley, MA (US); Nhan Le, Southborough, MA (US); Wei Kang, Newton Center, MA (US)

(21) Appl. No.: 18/885,560

(22) Filed: Sep. 13, 2024

Related U.S. Application Data
(60) Provisional application No. 63/582,461, filed on Sep. 13, 2023.

(51) Int. Cl. A61C 9/00; G06T 7/00

(52) U.S. Cl. CPC A61C 9/0053; G06T 7/0012; G06T 2207/10101; G06T 2207/30036

(57) ABSTRACT

Embodiments of the present invention generate comprehensive and accurate three-dimensional (3D) models of dental anatomy from acquired Cone Beam Computed Tomography (CBCT) data of the dental anatomy and acquired Optical Coherence Tomography (OCT) data of the dental anatomy. Generating the 3D model of the dental anatomy includes identifying one or more pathology volumes within the dental anatomy, based on the CBCT data and the OCT data.

### CROSS REFERENCE TO RELATED APPLICATIONS

This application claims a benefit of U.S. Provisional Patent Application No. 63/582,461, filed Sep. 13, 2023, titled “SYSTEMS AND METHODS FOR ORTHODONTIC IMAGING BASED ON COHERENCE TOMOGRAPHY SCANNING (OCT) AND CONE BEAM CT (CBCT),” the entire contents of which are hereby incorporated by reference herein, for all purposes.

### BACKGROUND

### Technical Field

The invention relates to dental imaging, and, in specific examples, to methods and systems for dental imaging that generate a three-dimensional model of a dental anatomy.

### Related Art

Dental caries is a common disease that affects more than 90% of American adults. Despite advances in preventive measures, dental caries continues to be a primary reason for invasive treatment to restore teeth. Over 35% of Americans do not see a dentist in any given year, and the United States Centers for Disease Control and Prevention (CDC) indicates that about 28% have untreated tooth decay. Research indicates that, of the patients who visit dentists, patient acceptance of an ideal dental treatment plan occurs only 28% of the time. It is believed that the main reasons for this low acceptance rate are: cost of care, inconvenience of multiple and lengthy dental appointments, and poor case acceptance by both patients and insurance carriers.

Avoiding dentists for these reasons usually results in dental disease progression, periodontal disease, and other oral problems, e.g., lack of detection of oral cancers, which have been associated with numerous adverse medical impacts, including eating disorders, speech difficulties, poor social interactions, reduced employment potential, and an increased risk of systemic diseases, such as diabetes, cardiovascular disease, such as stroke and heart attacks, and Alzheimer's disease. Health issues resulting from poor oral health have been shown to culminate in over $45B of lost productivity in the United States and over 34M lost school hours for young adults. There is, therefore, a critical unmet need for affordable and efficient dental health care.

To address these problems and increase access to dental care, a means is needed to lower treatment costs, shorten appointments, and improve case acceptance by patients and insurers. Treatment costs can be lowered, and appointments can be shortened, by improving prevention. Increased early and accurate diagnosis would improve preventative care.

In early stages of dental caries, loss of minerals in a tooth can be reversed with sufficient supply of calcium, phosphate, and fluoride ions in the mouth. These ions help to re-mineralize the tooth. Early and accurate diagnosis of dental caries lowers dental treatment costs, as it allows for the use of non-invasive treatment methods to prevent or forestall the onset and progression of the disease.

To improve case acceptance by patients and insurers, a more sensitive and specific imaging modality that is easy to read by both patients and insurers is needed. Today, patients are unaccustomed to interpreting two-dimensional (2D) radiographs and thus are unable to independently verify the need for care without a provider's interpretation. Three-dimensional (3D) radiographs, such as Cone Beam Computed Tomography (CBCT), circumvent this problem, and may improve overall case acceptance by 10%. However, radiographs have their own inherent limitations, including low sensitivity and specificity. This often creates discrepancies between providers and payers, resulting in patients not being covered by insurance, and discrepancies between providers, which lowers trust in the profession and, thus, reduces case acceptance.

### SUMMARY OF EMBODIMENTS

A need exists for methods and systems for dental imaging that enable a comprehensive and accurate 3D model of a dental anatomy to be generated.

The present disclosure addresses this need by providing a dental imaging system. The system includes at least one processor and data storage. Instructions are stored on the data storage. When executed by the at least one processor, the instructions cause the system to perform actions. The actions include receiving Optical Coherence Tomography (OCT) data of the dental anatomy. Cone Beam Computed Tomography (CBCT) data of the dental anatomy is received. A three-dimensional (3D) model of the dental anatomy is generated, based on the CBCT and OCT data. Generating of the 3D model of the dental anatomy includes identifying one or more pathology volumes within the dental anatomy, based on the CBCT and OCT data.

Optionally, in any embodiment, at least some of the one or more pathology volumes each correspond to a caries, a fracture, or a resorption.

Optionally, in any embodiment, identifying one or more pathology volumes within the dental anatomy includes determining, based on the OCT data, one or more estimated pathology volumes and determining, based on the CBCT data, and the one or more estimated pathology volumes, the one or more pathology volumes within the dental anatomy.

Optionally, in any embodiment, generating the three-dimensional (3D) model of the dental anatomy includes determining, based on the OCT data, external surfaces for one or more teeth of the dental anatomy.

Optionally, in any embodiment in which generating the three-dimensional (3D) model of the dental anatomy includes determining, based on the OCT data, external surfaces for one or more teeth of the dental anatomy, generating the three-dimensional (3D) model of the dental anatomy includes determining, based on the CBCT data, external surfaces for the one or more teeth of the dental anatomy.

Optionally, in any embodiment in which generating the three-dimensional (3D) model of the dental anatomy includes determining, based on the CBCT data, external surfaces for the one or more teeth of the dental anatomy, generating the three-dimensional (3D) model of the dental anatomy includes registering the CBCT data relative to the OCT data, based on the external surfaces for the one or more teeth, as determined using the OCT and CBCT data.

Optionally, in any embodiment in which generating the three-dimensional (3D) model of the dental anatomy includes determining, based on the CBCT data, external surfaces for the one or more teeth of the dental anatomy, the actions includes identifying at least one of an artifact region, or a missing region in the external surfaces for the one or more teeth determined using the OCT data; and correcting the at least one of the artifact region, or the missing region, based on a corresponding at least one region of the external surface for the one or more teeth determined using the CBCT data.

Optionally, in any embodiment in which generating the three-dimensional (3D) model of the dental anatomy includes determining, based on the CBCT data, external surfaces for the one or more teeth of the dental anatomy, generating the three-dimensional (3D) model of the dental anatomy includes determining external surfaces for one or more teeth of the dental anatomy based on a weighted function of: the external surfaces for the one or more teeth of the dental anatomy, as determined using the OCT data; and the external surfaces for the one or more teeth of the dental anatomy, as determined using the CBCT data, wherein a weighting for the external surfaces, as determined using the OCT data, is greater than a weighting for the external surfaces, as determined using the CBCT data.

Optionally, in any embodiment, the generating of the 3D model of the dental anatomy includes identifying gingiva of the dental anatomy based on the OCT data.

Optionally, in any embodiment, the generating of the 3D model of the dental anatomy includes identifying, based on the CBCT data, at least one of: a root for at least one tooth of the dental anatomy; a pulp cavity for at least one tooth of the dental anatomy; or a jawbone of the dental anatomy.

Optionally, in any embodiment, the actions further include: receiving color image data of the dental anatomy; and mapping the color image data onto external surfaces of the 3D model of the dental anatomy.

Optionally, in any embodiment, the actions further include generating a visualization of the 3D model of the dental anatomy, and displaying the visualization to a user.

Another embodiment of the present invention provides a dental imaging system. The system includes at least one processor and data storage. Instructions are stored on the data storage. When executed by the at least one processor, the instructions cause the system to perform actions including: receiving Optical Coherence Tomography (OCT) data of the dental anatomy; generating, based on the OCT data, a three-dimensional (3D) model of the dental anatomy, wherein the generating of the 3D model of the dental anatomy comprises identifying one or more estimated pathology volumes within the dental anatomy, based on the OCT data; and for each of the one or more estimated pathology volumes, predicting whether an actual pathology volume extends deeper within a corresponding tooth of the dental anatomy than the estimated pathology volume in question.

Optionally, in any embodiment that provides a dental imaging system, an indication is displayed to a user that Cone Beam Computed Tomography (CBCT) imaging of the dental anatomy should be carried out.

Optionally, in any embodiment that displays an indication to a user that Cone Beam Computed Tomography (CBCT) imaging of the dental anatomy should be carried out, the indication specifies one or more teeth having one or more pathology volumes that are each predicted to extend deeper within a corresponding tooth of the dental anatomy than the corresponding estimated pathology volume.

Optionally, in any embodiment that provides a dental imaging system, Cone Beam Computed Tomography (CBCT) imaging of the dental anatomy is performed.

Optionally, in any embodiment that provides a dental imaging system, at least some of the one or more pathology volumes each correspond to a caries, a fracture, or a resorption.

Yet another embodiment of the present invention provides a method of imaging a dental anatomy. The method includes receiving Optical Coherence Tomography (OCT) data of the dental anatomy; receiving Cone Beam Computed Tomography (CBCT) data of the dental anatomy; and generating a three-dimensional (3D) model of the dental anatomy based on the CBCT and OCT data. The generating of the 3D model of the dental anatomy includes identifying one or more pathology volumes within the dental anatomy, based on the CBCT and OCT data.

Optionally, in any method of imaging a dental anatomy that includes receiving Optical Coherence Tomography (OCT) data of the dental anatomy, receiving Cone Beam Computed Tomography (CBCT) data of the dental anatomy, and generating a three-dimensional (3D) model of the dental anatomy based on the CBCT and OCT data, identifying one or more pathology volumes within the dental anatomy includes: determining, based on the OCT data, one or more estimated pathology volumes; and determining, based on the CBCT data, and the one or more estimated pathology volumes, the one or more pathology volumes within the dental anatomy.

Optionally, in any method of imaging a dental anatomy that includes receiving Optical Coherence Tomography (OCT) data of the dental anatomy, receiving Cone Beam Computed Tomography (CBCT) data of the dental anatomy, and generating a three-dimensional (3D) model of the dental anatomy based on the CBCT and OCT data, generating the three-dimensional (3D) model of the dental anatomy includes: determining, based on the OCT data, external surfaces for one or more teeth of the dental anatomy; determining, based on the CBCT data, external surfaces for the one or more teeth of the dental anatomy; and registering the CBCT data relative to the OCT data, based on the external surfaces for the one or more teeth, as determined using the OCT and CBCT data.

Optionally, in any method of imaging a dental anatomy that includes receiving Optical Coherence Tomography (OCT) data of the dental anatomy, receiving Cone Beam Computed Tomography (CBCT) data of the dental anatomy, and generating a three-dimensional (3D) model of the dental anatomy based on the CBCT and OCT data, generating the three-dimensional (3D) model of the dental anatomy includes: determining, based on the OCT data, external surfaces for one or more teeth of the dental anatomy; and determining, based on the CBCT data, external surfaces for the one or more teeth of the dental anatomy. The method further includes: identifying at least one of an artifact region, or a missing region in the external surfaces for the one or more teeth determined using the OCT data; and correcting the at least one of the artifact region, or the missing region, based on a corresponding at least one region of the external surface for the one or more teeth determined using the CBCT data.

An embodiment of the present invention provides a method of imaging a dental anatomy. The method includes receiving Optical Coherence Tomography (OCT) data of the dental anatomy; generating, based on the OCT data, a three-dimensional (3D) model of the dental anatomy, wherein the generating of the 3D model of the dental anatomy comprises identifying one or more estimated pathology volumes within the dental anatomy, based on the OCT data; and for each of the one or more estimated pathology volumes, predicting whether an actual pathology volume extends deeper within a corresponding tooth of the dental anatomy than the estimated pathology volume in question.

Yet another embodiment of the present invention provides a non-transitory computer readable storage medium. Instructions are stored on the medium. When executed by at least one processor of a dental imaging system, the instructions cause the system to perform actions. The action include: receiving Optical Coherence Tomography (OCT) data of the dental anatomy; receiving Cone Beam Computed Tomography (CBCT) data of the dental anatomy; and generating a three-dimensional (3D) model of the dental anatomy based on the CBCT and OCT data. The generating of the 3D model of the dental anatomy includes identifying one or more pathology volumes within the dental anatomy, based on the CBCT and OCT data.

Optionally, in any embodiment that includes a non-transitory computer readable storage medium, identifying one or more pathology volumes within the dental anatomy includes:
determining, based on the OCT data, one or more estimated pathology volumes; and
determining, based on the CBCT data, and the one or more estimated pathology volumes, the one or more pathology volumes within the dental anatomy.

Optionally, in any embodiment that includes a non-transitory computer readable storage medium, generating the three dimensional (3D) model of the dental anatomy includes: determining, based on the OCT data, external surfaces for one or more teeth of the dental anatomy; determining, based on the CBCT data, external surfaces for the one or more teeth of the dental anatomy; and registering the CBCT data relative to the OCT data, based on the external surfaces for the one or more teeth, as determined using the OCT and CBCT data.

Optionally, in any embodiment that includes a non-transitory computer readable storage medium, generating the three-dimensional (3D) model of the dental anatomy includes: determining, based on the OCT data, external surfaces for one or more teeth of the dental anatomy; and determining, based on the CBCT data, external surfaces for the one or more teeth of the dental anatomy. The method further includes: identifying at least one of an artifact region, or a missing region in the external surfaces for the one or more teeth determined using the OCT data; and correcting the at least one of the artifact region, or the missing region, based on a corresponding at least one region of the external surface for the one or more teeth determined using the CBCT data.

An embodiment of the present invention provides a non-transitory computer readable storage medium. Instructions are stored on the storage medium. When executed by at least one processor of a dental imaging system, the instructions cause the system to perform actions. The actions include: receiving Optical Coherence Tomography (OCT) data of the dental anatomy; generating, based on the OCT data, a three-dimensional (3D) model of the dental anatomy, wherein the generating of the 3D model of the dental anatomy comprises identifying one or more estimated pathology volumes within the dental anatomy, based on the OCT data; and for each of the one or more estimated pathology volumes, predicting whether an actual pathology volume extends deeper within a corresponding tooth of the dental anatomy than the estimated pathology volume in question.

### BRIEF DESCRIPTION OF THE DRAWINGS

The invention will be more fully understood by referring to the following Detailed Description of Specific Embodiments in conjunction with the Drawings, of which:

FIG. 1 is a flow diagram that schematically illustrates a method of imaging a dental anatomy according to an example of a first aspect of this disclosure.

FIG. 2 is a flow diagram that schematically illustrates operations for identifying pathology volumes within a dental anatomy that may be carried out as part of a specific example of the method of FIG. 1.

FIG. 3 is an example of a visualization of a 3D model of a dental anatomy that is produced using the method of FIG. 1.

FIG. 4 is an anatomical diagram of a tooth.

FIG. 5 shows a visualization of the same 3D model as FIG. 3, but from a more distant viewpoint.

FIGS. 6A and 6B show visualizations where color image data has been mapped onto the same 3D model as FIGS. 3 and 5.

FIG. 7 is a schematic block diagram that illustrates a dental imaging system according to an example of a further aspect of this disclosure.

FIG. 8 is a flow diagram illustrating a method of imaging a dental anatomy according to an example of a still further aspect of this disclosure.

FIG. 9 is a schematic block diagram that illustrates a dental imaging system according to an example of a still further aspect of this disclosure.

### DETAILED DESCRIPTION OF SPECIFIC EMBODIMENTS

Embodiments of the present invention provide methods and systems for dental imaging that enable a comprehensive and accurate 3D model of a dental anatomy to be generated.

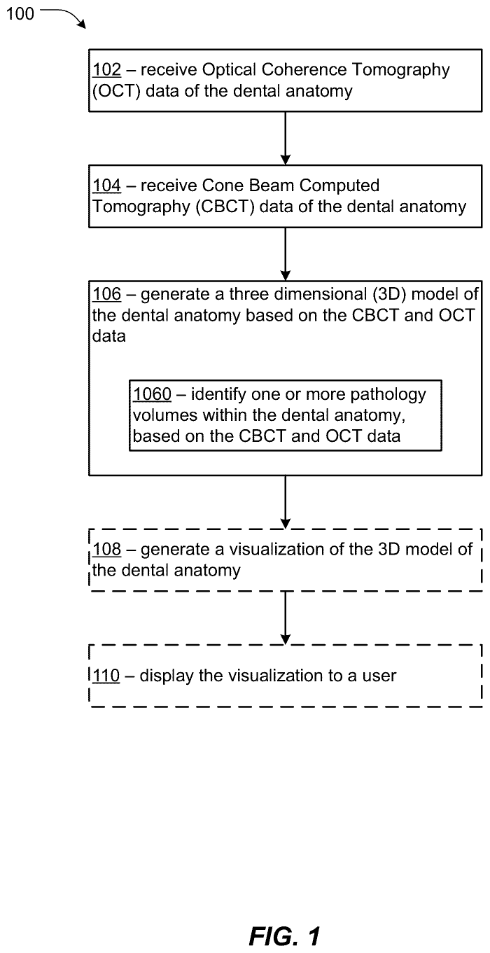

Reference is directed firstly to FIG. 1, which is a flow diagram illustrating a method 100 of imaging a dental anatomy according to an example of a first aspect of this disclosure.

As shown, method 100 comprises a step 102 of receiving Optical Coherence Tomography (OCT) data of the dental anatomy. Such data may be generated by carrying out a scan of a patient's teeth using an OCT scanner as described in WO2022/212507A1 and/or as described in WO2024/054937A1, for example. In some examples, method 100 may comprise an additional step (not shown) of carrying out a scan, with an OCT scanning device, to generate the OCT data.

The OCT data may, for example, comprise (or be in the form of) a plurality of OCT volumetric data sets, where each of the OCT volumetric data sets was produced by a traversal of a scanning pattern by a beam emitted from the OCT scanning system towards the dental anatomy. Each volumetric data set may, for example, define a cloud of points, distributed over a 3D volume, where each point in the point cloud is associated with a respective value that is representative of the scattering of light emitted by the OCT scanner, at that spatial location. The scanning pattern can, for instance, be a deterministic, smooth, two-dimensional scan pattern, such as a Lissajous pattern.

It should be appreciated that each of the plurality of OCT volumetric data sets corresponds to the OCT scanning system 1000 and the dental anatomy being in a respective relative arrangement (i.e., a respective position and orientation). By capturing a plurality of OCT volumetric data sets at various relative arrangements (i.e., with different positions and/or orientations) a comprehensive 3D model of the dental anatomy can be generated. As will be appreciated, to capture data sets at various different relative arrangements, the OCT scanning system 1000 (in particular, a scanning device thereof) may be moved by the operator relative to the dental anatomy. Depending on the particular design of the OCT scanning system 1000, such movement could be carried out manually, or using a robotic manipulator (e.g., a robotic arm). However, with other designs of the OCT scanning system 1000, the dental anatomy could instead be moved relative to the OCT scanning system 1000, or both could be moved simultaneously relative to one another.

In other examples, the OCT data may have been previously processed to determine surfaces within the dental anatomy (e.g., the external surfaces of the teeth and gingiva) and/or to reconstruct full volumetric details of the interior of the dental anatomy.

Returning to FIG. 1, as shown, method 100 further comprises a step 104 of receiving Cone Beam Computed Tomography (CBCT) data of the same dental anatomy. As will be understood, such CBCT data can, for example, be generated by scanning a patient with a commercially available CBCT dental scanner, such as the Axeos™ Imaging System available from Dentsply Sirona. In some examples, method 100 may comprise an additional step (not shown) of carrying out a scan, with an CBCT scanning device, to generate the CBCT data.

The CBCT data may, for example, define a plurality of points distributed over a 3D volume and, for each such point, a respective value that is representative of the absorptivity of X-rays at that point.

It should be appreciated that, although FIG. 1 shows step 102 as occurring prior to step 104, this is by no means essential. In other examples, step 104 could occur prior to step 102, or both step 102 and step 104 could occur in parallel, so that they overlap in time.

As further shown in FIG. 1, method 100 additionally comprises a step 106 of generating a three-dimensional (3D) model of the dental anatomy based on the CBCT and OCT data. A 3D model generated using a combination of these imaging modalities can benefit from the high spatial resolution (currently about 1-20 μm) of OCT in the vicinity of the tooth surface, the high sensitivity and specificity of OCT for detecting caries, tooth decay, and other pathologies (such as cracks and resorptions), and the ability of OCT to identify and distinguish gingival tissue, and can also benefit from the ability of CBCT to see deep into tooth and bone, allowing deep caries, pulp cavities and roots, and the jawbone to be identified.

As indicated in FIG. 1, the generating of the 3D model in step 106 comprises identifying one or more pathology volumes within the dental anatomy, based on the CBCT and OCT data. As will be appreciated from the discussion above, the OCT data may particularly (but not exclusively) be used to identify pathology volumes (or portions thereof) in the vicinity of the surfaces of the teeth, whereas the CBCT data may particularly (but not exclusively) be used to identify pathology volumes (or portions thereof) deeper within the teeth. In some examples, the pathology volumes correspond to caries; however, in other examples, the pathology volumes could correspond more generally to caries, fractures/cracks, and/or resorptions, for example.

The ability of OCT and CBCT to identify different anatomical structures can be leveraged to generate a particularly comprehensive 3D model of the dental anatomy. For instance, in some cases, the generating of the 3D model in step 106 may comprise identifying gingiva of the dental anatomy based on the OCT data (but not, for example, the CBCT data). Similarly, in some cases, the generating of the 3D model in step 106 may comprise identifying, based on the CBCT data (but not, for example, the OCT data) the roots, and/or the pulp cavities of teeth, and/or the jawbone.

It should be appreciated that the identifying of pathology volumes in step 106 may utilize various algorithms, such as segmentation and/or object identification algorithms. Hence, or otherwise, step 106 may, for instance, utilize thresholding, edge detection, and/or neural networks. Suitable neural networks may include UNet, VNet, transformers, mask r-cnn, graph neural networks, LSTM, Fast SCNN, YOLO, or RDUNet. An algorithm may, in some examples, take both the OCT and the CBCT data as inputs to identify pathology volumes; in other examples, separate segmentation and/or object identification algorithms may act on the OCT and the CBCT data respectively, with the results of the separate algorithms being combined to arrive at a final determination of pathology volumes within the dental anatomy.

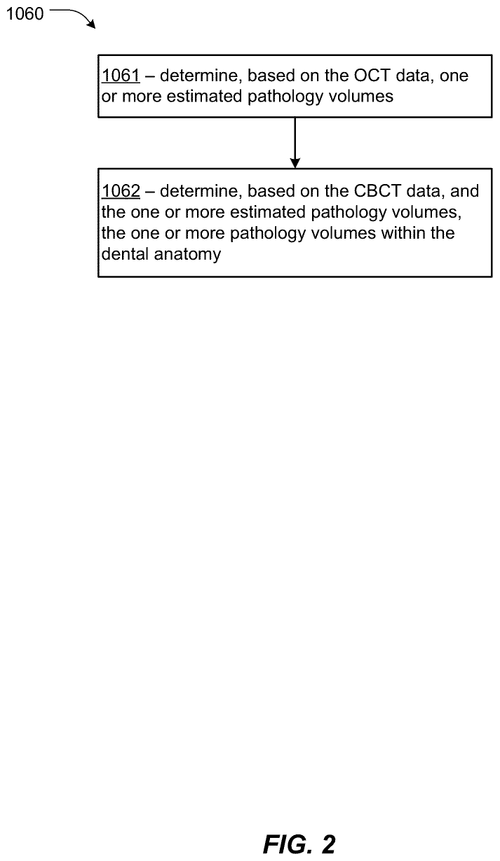

Referring now to FIG. 2, it should be noted that, in some examples, the identifying in step 1060 (FIG. 1) of the one or more pathology volumes within the dental anatomy may comprise two (or more) (sub) steps. For instance, as illustrated in FIG. 2, the OCT data might be used, in a first step 1061, to determine estimated pathology volumes. The CBCT data may then be used, in a second step 1062, to determine final pathology volumes, e.g., the pathology volumes that form part of the final 3D model of the dental anatomy. Such an approach may leverage OCT's high sensitivity and specificity for detecting caries, tooth decay, and other pathologies, by using the OCT data to, for example, accurately identify and locate the estimated pathology volumes, and may leverage CBCT's ability to see deeper into the teeth, by using the CBCT data to, for example, determine the depth of the estimated pathology volumes that were identified using the OCT data. In addition, or instead, the OCT data may be regarded as resolving ambiguities that might arise if pathology volumes were identified solely using the CBCT data.

As will be appreciated, the generating of the 3D model of the dental anatomy based on the CBCT and OCT data in step 106 typically comprises determining external surfaces for the teeth. In some examples, the CBCT data and the OCT data are processed separately to provide respective determinations of the 3D shape of the external surfaces. Such respective determinations of the external surfaces may be utilized in various ways. For instance, in some cases, the respective determinations can be used to register the OCT data relative to the CBCT data, i.e., they can be used to determine a spatial transformation (which may be rigid, or non-rigid, depending on the particular implementation) between a frame of reference for the OCT data and a frame of reference for the CBCT data. This approach for registration is believed to be particularly effective because the external surfaces of the teeth provide especially sharp and well-defined 3D features in both OCT and CBCT data.

In the same, or other cases, the external surfaces determined using the CBCT data may be used to correct for artifacts or missing regions in the external surfaces determined using the OCT data. In general, it may be preferable to use the external surfaces as determined using the OCT data, given the significantly higher accuracy of OCT imaging; however, OCT imaging can suffer from artifacts and missing regions, particularly when produced using a scanning device that must be moved relative to the dental anatomy. Even though the CBCT data provides a lower accuracy determination of the external surfaces, it is still suitable for correcting for such artifact regions and/or missing regions.

In the same, or other cases, the external surfaces for the teeth in the final 3D model of the dental anatomy may be determined based on a weighted function (e.g., a weighted average) of the respective determinations based on the OCT data and the CBCT data. Given the higher accuracy of OCT imaging, the weighting for the surfaces determined using OCT data will be greater than that for the surfaces determined using CBCT data.

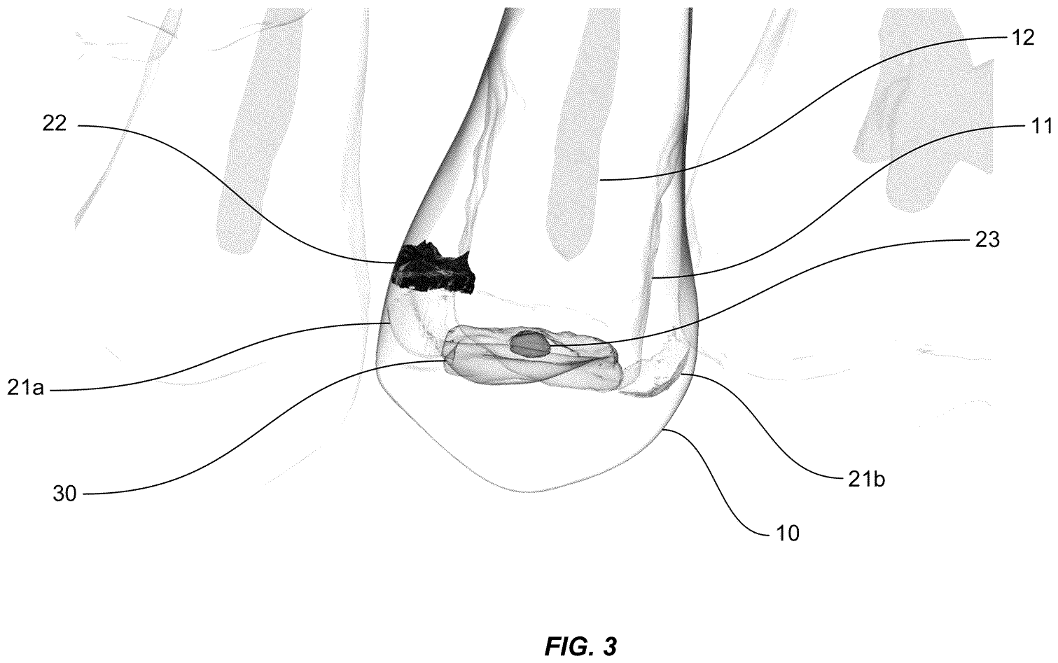

Referring again to FIG. 1, as shown, the method 100 may optionally further comprise a step 108 of generating a visualization of the 3D model of the dental anatomy, and a step 110 of displaying the visualization to a user. This visualization can be displayed using a display screen, VR/AR headset, or the like, to an operator of a system that carries out method 100 (such as a dentist, dental assistance, or other dental professional), and/or may be displayed to the patient whose dental anatomy has been imaged to generate the OCT and CBCT data. The visualization can help such a dental professional provide a compelling case presentation to the patient, as the patient can readily understand, for example, the extent of pathologies (such as caries or fractures) within their teeth, particularly in relation to important anatomical structures of the teeth, such as the dentin, or pulp cavity. The visualization can be generated using any suitable computer graphics process, such as a 3D rendering process. An example of a visualization is illustrated in FIG. 3, which indicates a particular tooth 10 within the dental anatomy for which pathology volumes 21a, 21b, 22, 23 have been identified.

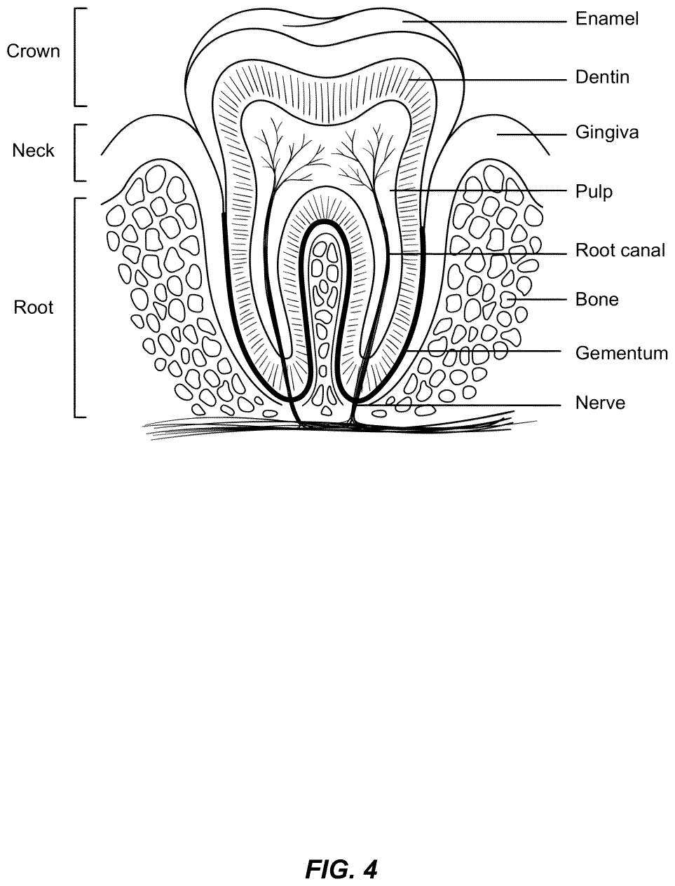

A user (i.e., the dental professional or the patient) may be presented with a user interface that allows him/her to selectively highlight features of the dental anatomy that have been identified during the generating of the 3D model in step 106. For example, the user can choose to highlight specific pathologies, such as fractures 21a, 21b, caries 22, and/or voids 23, which are visible in the visualization of FIG. 3. Equally, the user may use the user interface to highlight previously applied restorations 30, and/or to highlight anatomical features of the tooth, such as the dentin 11 or pulp 12 of a tooth 10. (To aid the reader's understanding, such anatomical features are illustrated in FIG. 4, which is an anatomical diagram of a tooth.)

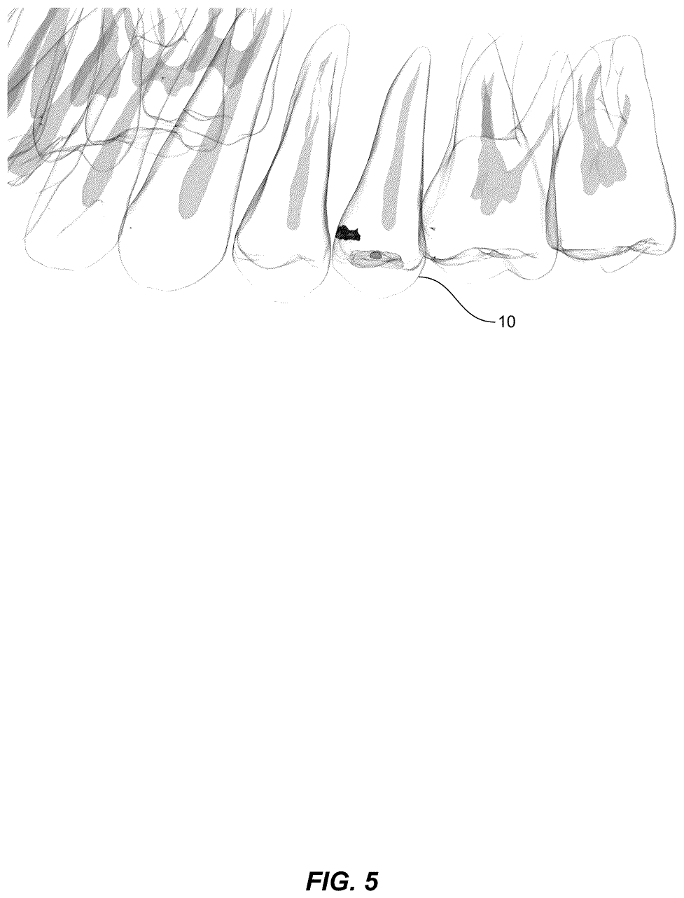

The user interface may also (or instead) enable the user to control the viewpoint for the visualization, rotating and/or moving the 3D model to a desired arrangement (i.e., a desired position and orientation) that allows features of interest within the dental anatomy to be seen more clearly. As an example, FIG. 5 shows a visualization of the same 3D model as FIG. 3, but from a more distant viewpoint, so that more of the teeth in proximity to tooth 10 are visible.

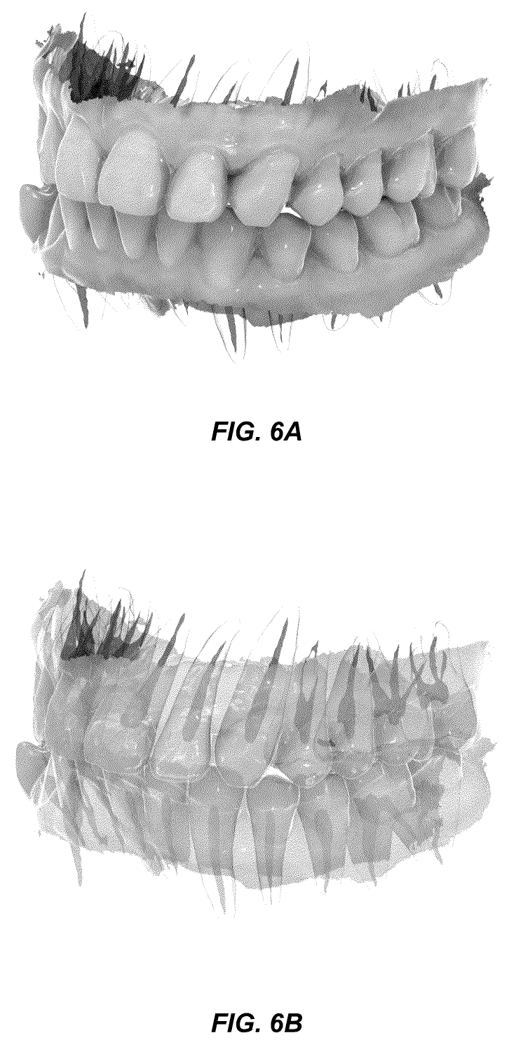

In some examples, method 100 may further comprise receiving color image data of the dental anatomy. Such color image data may, for example, be mapped onto the external surfaces 3D model of the dental anatomy (e.g., the external surfaces of the teeth and the external surfaces of the gingiva), to provide a more compelling and/or intelligible visualization of the dental anatomy. FIG. 6A shows an example of a visualization of a 3D model, which has had color image data mapped onto its external surfaces. As is apparent, the gingiva can easily be distinguished from the teeth.

Suitable color image data may, for example, be captured using a camera that is mounted on and/or fixed to the OCT scanning device. In a particular example, the camera may be configured (e.g., by suitable calibration and/or positioning) such that each a-line in the OCT-scan corresponds to a respective pixel in the image captured by the camera. This allows the color image data generated by the camera to be easily mapped onto the 3D model generated in step 106.

It is, however, by no means essential that the camera which captures the color image data be mounted on and/or fixed to the OCT scanning device. Instead, the camera could, for example, be mounted on and/or fixed to a further scanning device, such as an intraoral scanner, that is capable of determining its spatial arrangement relative to the dental anatomy. In such a case, the 3D model of the dental anatomy generated by the scanning device can easily be registered with the 3D model generated in step 106, which allows the color image data from the camera to be mapped onto the 3D model generated in step 106.

Particularly (but not exclusively) where the visualization uses color image data mapped onto the 3D model of the dental anatomy, the user interface may allow the user to alter the opacity of the teeth in the visualization so that internal features can be seen more clearly. This is illustrated by FIGS. 6A and 6B which show, respectively, a visualization where the teeth are fully opaque (FIG. 6A) and a visualization where the opacity of the teeth has been reduced (FIG. 6B).

It should be appreciated that it is by no means essential that a visualization of the 3D model be generated and hence (or otherwise) step 108 and step 110 are optional. In some examples, a visualization of the 3D model may not be generated because the 3D model of the dental anatomy may be used solely by automated processes. For instance, the 3D model could be utilized to plan a cutting pattern for a robotic dental system (such as the systems described in WO2019/215511A2, WO2019/215512A1, or U.S. application Ser. No. 18/656,502).

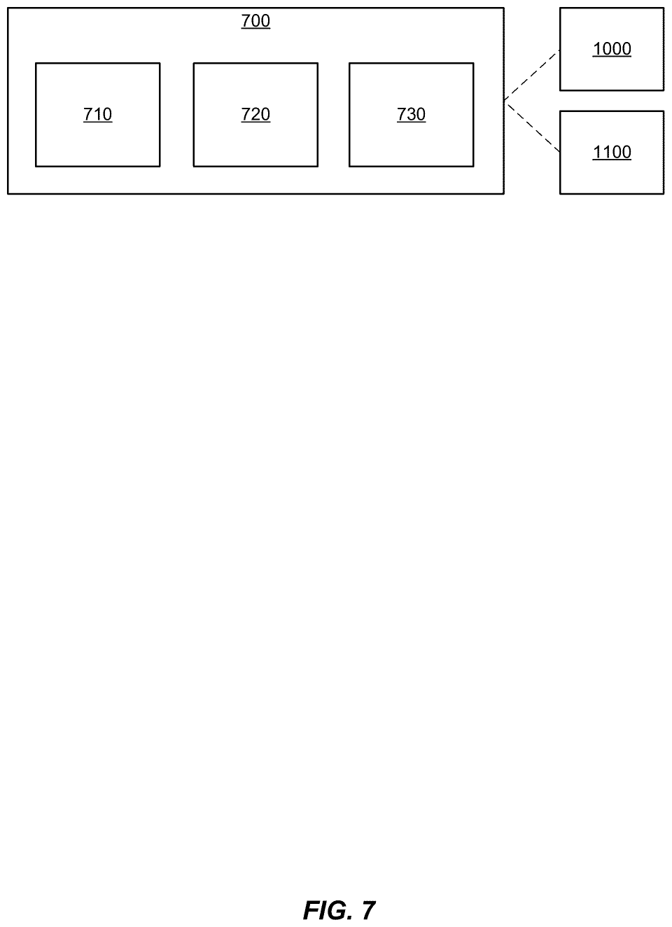

Reference is now directed to FIG. 7, which is a schematic block diagram illustrating a system 700 according to an example of a further aspect of this disclosure. As shown, the system 700 comprises at least one processor 710 and data storage 720. The data storage 720 stores instructions that, when executed by at least one processor 710, cause the system 700 to carry out one or more of the examples of method 100 described herein. The at least one processor 710 may be of any suitable type. Particularly (but not exclusively), where the generating of the 3D model in step 106 utilizes neural network algorithms, the at least one processor 710 may comprise at least one graphical processing unit (GPU).

As also shown in FIG. 7, the system 700 can be in data communication with an OCT scanning system 1000 (for example as described in WO2022/212507A1 and/or as described in WO2024/054937A1), from which the system 700 receives the series of OCT volumetric data sets. In addition, as also shown in FIG. 7, the system 700 can be in data communication with a CBCT scanning system 1100.

In some examples, the system 700 might be configured as a cart, which carries computer hardware (including the at least one processor 710 and the data storage 720, as well as, for example, a display screen 730 for displaying the visualization generated in step 108) and a power supply (not shown) therefore. The cart may further include a physical connection port for data connection to the OCT scanning system 1000 and/or the CBCT scanning system 1100. It is, of course, not essential that system 700 is configured as a cart, and, in other examples, the system 700 could simply be configured as a general purpose computer, provided that the processor(s) thereof have sufficient processing power to carry out a method 100 as described herein.

In still other examples, an OCT scanning system 1000 and/or a CBCT scanning system 1100 may form part of the system 700 provided to the end user.

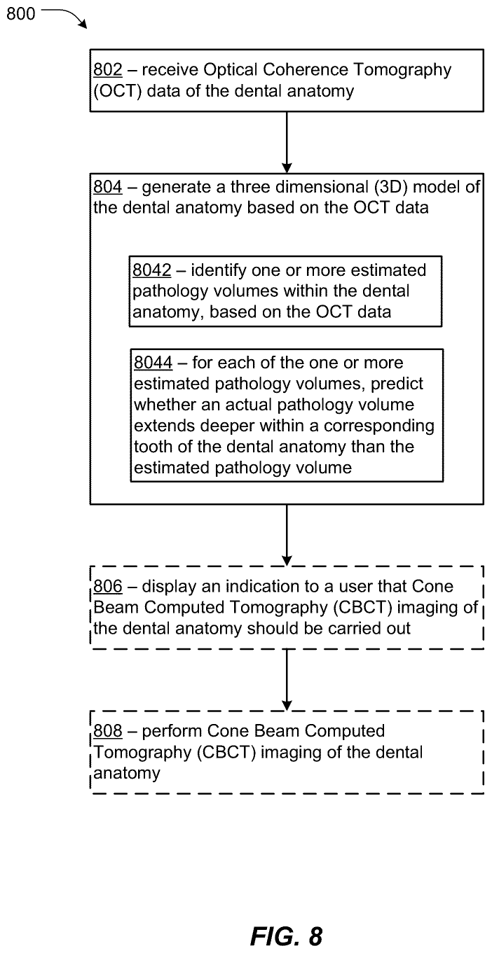

Reference is now directed to FIG. 8, which is a flow diagram illustrating a method 800 of imaging a dental anatomy according to an example of a further aspect of this disclosure.

As shown, method 800 comprises a step 802 of receiving Optical Coherence Tomography (OCT) data of the dental anatomy. As with method 100, such OCT data may be generated by carrying out a scan of a patient's teeth using an OCT scanner as described in WO2022/212507A1 and/or as described in WO2024/054937A1, for example. In some examples, method 800 may comprise an additional step (not shown) of carrying out a scan, with an OCT scanning device, to generate the OCT data.

As with method 100, the OCT data may, for example, comprise (or be in the form of) a plurality of OCT volumetric data sets, where each of the OCT volumetric data sets was produced by a traversal of a scanning pattern by a beam emitted from the OCT scanning system towards the dental anatomy. Each volumetric data set may, for example, define a cloud of points, distributed over a 3D volume, where each point in the point cloud is associated with a respective value that is representative of the attenuation of light emitted by the OCT scanner, at that spatial location. The scanning pattern can, for instance, be a deterministic, smooth, two-dimensional scan pattern, such as a Lissajous pattern.

Similarly, as with method 100, each of the plurality of OCT volumetric data sets corresponds to the OCT scanning system 1000 and the dental anatomy being in a respective relative arrangement (i.e., a respective position and orientation). As discussed above with reference to method 100, to capture data sets at various different relative arrangements, the OCT scanning system 1000 (in particular, a scanning device thereof) may be moved by the operator relative to the dental anatomy. Depending on the particular design of the OCT scanning system 1000, such movement could be carried out manually, or using a robotic manipulator (e.g., a robotic arm). However, with other designs of the OCT scanning system 1000, the dental anatomy could instead be moved relative to the OCT scanning system 1000, or both could be moved simultaneously relative to one another.

Returning to FIG. 8, as shown, method 800 further comprises a step 804 of generating a three-dimensional (3D) model of the dental anatomy based on the OCT data. As also shown in FIG. 8, the generating of the 3D model comprises a step 8042 of identifying one or more estimated pathology volumes within the dental anatomy, based on the OCT data, and a step 8044 of predicting, for each of the estimated pathology volumes, whether an actual pathology volume extends deeper within a corresponding tooth of the dental anatomy than the estimated pathology volume in question. As with step 106 of method 100, the identifying of pathology volumes in step 8042 may utilize various algorithms, such as segmentation and/or object identification algorithms. Hence, or otherwise, step 8042 may, for instance, utilize thresholding, and/or neural networks. Suitable neural networks may include UNet, VNet, transformers, mask r-cnn, graph neural networks, LSTM, Fast SCNN, YOLO, or RDUNe.

As to step 8044, the predicting of whether an actual pathology volume extends deeper within a corresponding tooth of the dental anatomy than the corresponding estimated pathology volume may utilize various techniques and/or algorithms. In a simple example, step 8044 may comprise determining whether a substantial portion of an estimated pathology volume has a depth beneath the tooth surface that is approximately equal to a known sensing depth limit for the OCT scanner. In a more complex example, artificial intelligence and/or neural network algorithms may be utilized to determine whether the actual pathology volume extends deeper into the tooth. Such algorithms may, for example, be trained using OCT scan data where the estimated pathology volumes have been segmented and using ground truth data indicating whether (or not) the actual pathology volumes extend deeper into the tooth.

By predicting whether actual pathology volumes extend deeper into the tooth/teeth, it is possible to determine whether carrying out a CBCT scan of the dental anatomy is likely to be appropriate. Consequently, CBCT scans can be avoided where they are unlikely to provide a more complete picture of the pathologies within a patient's dental anatomy. This may, in turn, reduce the average visit time for patients and/or may reduce the amount of ionizing radiation that patients are, on average, exposed to.

Referring once more to FIG. 8, as shown, method 800 optionally comprises a step 806 of displaying an indication to a user that Cone Beam Computed Tomography (CBCT) imaging of the dental anatomy should be carried out. Such an indication can, for example, be displayed on a graphical user interface on a display screen. Moreover, such an indication may be displayed when, in step 8044, it is predicted that, for one or more of the pathology volumes estimated using OCT, the corresponding, actual pathology volumes likely extend deeper into the teeth. In such cases, a CBCT scan can be used to determine the actual extent of the pathology volumes, for example as described above with reference to FIGS. 1-6B.

As also shown in FIG. 8, method 800 may in addition, or instead, comprise a step 808 of actually performing such CBCT imaging of the dental anatomy. In particular, such CBCT imaging may be carried out where it is predicated in step 8044 that actual pathology volumes extend deeper within the corresponding tooth or teeth of the dental anatomy than the corresponding estimated pathology volumes. The CBCT imaging data can then be used to update the 3D model of the dental anatomy (particularly, but not exclusively, including the pathology volumes) in substantially the same way as described above with reference to FIGS. 1-6B.

In some cases, such CBCT imaging may be carried out using a CBCT scanner operated by a human. However, in other cases, the CBCT imaging could be carried out autonomously, e.g., by a robot. Particularly (but not exclusively) where the CBCT imaging is carried out autonomously, it may not be necessary for method 800 to include step 806 of display an indication to a user that Cone Beam Computed Tomography (CBCT) imaging of the dental anatomy should be carried out.

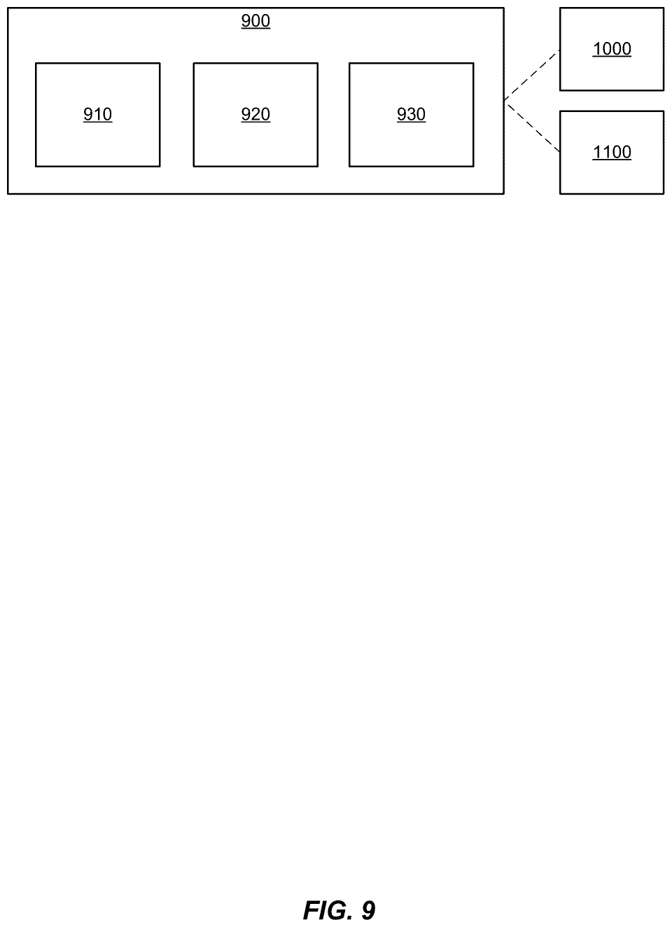

Reference is now directed to FIG. 9, which is a schematic block diagram illustrating a system 900 according to an example of a further aspect of this disclosure. As shown, the system 900 comprises at least one processor 910 and data storage 920. The data storage 920 stores instructions that, when executed by the at least one processor 910, cause the system 900 to carry out any of the examples of method 800 described herein. The at least one processor 910 may be of any suitable type. Particularly (but not exclusively), where the generating of the 3D model in step 804 utilizes neural network algorithms, the at least one processor 910 may comprise at least one graphical processing unit (GPU).

As also shown in FIG. 9, the system 900, like system 700 shown in FIG. 7, can be in data communication with an OCT scanning system 1000 (for example as described in WO2022/212507A1 and/or as described in WO2024/054937A1), from which the system 900 receives the series of OCT volumetric data sets. In addition, as also shown in FIG. 9, the system 900 can be in data communication with a CBCT scanning system 1100.

In such examples, the system 900 might be configured as a cart, which carries computer hardware (including the at least one processor 910 and the data storage 920, as well as, for example, a display screen 930 for displaying the indication generated in step 806 and/or for displaying a visualization of the 3D model generated in step 804). The cart may also carry a power supply (not shown) for supplying power to such computer hardware. The cart may further include a physical connection port for data connection to the OCT scanning system 1000 and/or the CBCT scanning system 1100. It is, of course, not essential that system 700 is configured as a cart, and, in other examples, the system 700 could simply be configured as a general-purpose computer, provided that the processor(s) thereof have sufficient processing power to carry out a method 800 as described herein.

In still other examples, an OCT scanning system 1000 and/or a CBCT scanning system 1100 may form part of the system 900 provided to the end user.

### Definitions

As used herein, the following term shall have the following meanings, unless context indicates otherwise.

The “arrangement” of an object refers to a combination of the object's position and orientation, so that when a reference is made to an object being in two different arrangements, this can mean that the object is in different positions, different orientations, or different positions and orientations, in the respective arrangements.

“Continually” means continuously or repeatedly, although not necessarily in perpetuity. The term continually encompasses periodically and occasionally. Continually generating a signal means generating a continuously varying signal over time or generating a series of (more than one) discrete signals over time. Continually generating a value, such as an error value, means generating a continuously varying value, such as an analog value represented by a continuously varying voltage, or generating a series of (more than one) discrete values over time, such as a series of digital or analog values.

While the invention is described through the above-described exemplary embodiments, modifications to, and variations of, the illustrated embodiments may be made without departing from the inventive concepts disclosed herein. For example, although specific parameter values, such as materials and dimensions, may be recited in relation to disclosed embodiments, within the scope of the invention, the values of all parameters may vary over wide ranges to suit different applications. Unless otherwise indicated in context, or would be understood by one of ordinary skill in the art, terms such as “about” mean within +20%.

As used herein, including in the claims, the term “and/or,” used in connection with a list of items, means one or more of the items in the list, i.e., at least one of the items in the list, but not necessarily all the items in the list. As used herein, including in the claims, the term “or,” used in connection with a list of items, means one or more of the items in the list, i.e., at least one of the items in the list, but not necessarily all the items in the list. “Or” does not mean “exclusive or.”

As used herein, including in the claims, an element described as being configured to perform an operation “or” another operation is met by an element that is configured to perform only one of the two operations. That is, the element need not be configured to operate in one mode in which the element performs one of the operations, and in another mode in which the element performs the other operation. The element may, however, but need not, be configured to perform more than one of the operations.

Although aspects of embodiments may be described with reference to flowcharts and/or block diagrams, functions, operations, decisions, etc. of all or a portion of each block, or a combination of blocks, may be combined, separated into separate operations or performed in other orders. References to a “module,” “operation,” “step,” and similar terms are for convenience and not intended to limit their implementation. All or a portion of each block, module, operation, step or combination thereof may be implemented as computer program instructions (such as software), hardware (such as combinatorial logic, Application Specific Integrated Circuits (ASICs), Field-Programmable Gate Arrays (FPGAs), processor or other hardware), firmware or combinations thereof.

The at least one processor 1010 of system 700 and/or the at least one processor 910 of system 900 may be a general-purpose processor, such as a central processing unit (CPU), a graphic processing unit (GPU), digital signal processor (DSP), a special purpose processor, etc., as appropriate, or combination thereof.

The data storage 720 of system 700 and/or the data storage 920 of system 900 may be random access memory (RAM), read-only memory (ROM), non-volatile memory (NVM), non-volatile random-access memory (NVRAM), flash memory or any other memory, or combination thereof, suitable for storing control software or other instructions and data. Instructions defining the functions of the present invention may be delivered to a processor in many forms, including, but not limited to, information permanently stored on tangible non-transitory non-writable storage media (e.g., read-only memory devices within a computer, such as ROM, or devices readable by a computer I/O attachment, such as CD-ROM or DVD disks), information alterably stored on tangible non-transitory writable storage media (e.g., floppy disks, removable flash memory and hard drives) or information conveyed to a computer through a communication medium, including wired or wireless computer networks. Moreover, while embodiments may be described in connection with various illustrative data structures, database schemas and the like, systems may be embodied using a variety of data structures, schemas, etc.

Disclosed aspects, or portions thereof, may be combined in ways not listed herein and/or not explicitly claimed. In addition, embodiments disclosed herein may be suitably practiced, absent any element that is not specifically disclosed herein. Accordingly, the invention should not be viewed as being limited to the disclosed embodiments.

As used herein, numerical terms, such as “first,” “second” and “third,” are used to distinguish respective elements, such as mirrors or traversals, from one another and are not intended to indicate any particular order or total number of mirrors or traversals in any particular embodiment. Thus, for example, a given embodiment may include only a second mirror and a third traversal.

What is claimed is:

1. A dental imaging system comprising:
at least one processor; and
data storage, on which is stored instructions that, when executed by the at least one processor, cause the system to perform actions comprising:
receiving Optical Coherence Tomography (OCT) data of the dental anatomy;
receiving Cone Beam Computed Tomography (CBCT) data of the dental anatomy; and
generating a three-dimensional (3D) model of the dental anatomy based on the CBCT and OCT data,
wherein the generating of the 3D model of the dental anatomy comprises identifying one or more pathology volumes within the dental anatomy, based on the CBCT and OCT data.

2. The system of claim 1, wherein at least some of the one or more pathology volumes each correspond to a caries, a fracture, or a resorption.

3. The system of claim 1, wherein identifying one or more pathology volumes within the dental anatomy comprises:
determining, based on the OCT data, one or more estimated pathology volumes; and
determining, based on the CBCT data, and the one or more estimated pathology volumes, the one or more pathology volumes within the dental anatomy.

4. The system of claim 1, wherein generating the three-dimensional (3D) model of the dental anatomy comprises determining, based on the OCT data, external surfaces for one or more teeth of the dental anatomy.

5. The system of claim 4, wherein generating the three-dimensional (3D) model of the dental anatomy comprises determining, based on the CBCT data, external surfaces for the one or more teeth of the dental anatomy.

6. The system of claim 5, wherein generating the three-dimensional (3D) model of the dental anatomy comprises registering the CBCT data relative to the OCT data, based on the external surfaces for the one or more teeth, as determined using the OCT and CBCT data.

7. The system of claim 5, wherein the actions comprise:
identifying at least one of an artifact region, or a missing region in the external surfaces for the one or more teeth determined using the OCT data; and
correcting the at least one of the artifact region, or the missing region, based on a corresponding at least one region of the external surface for the one or more teeth determined using the CBCT data.

8. The system of claim 5, wherein generating the three-dimensional (3D) model of the dental anatomy comprises determining external surfaces for one or more teeth of the dental anatomy based on a weighted function of: the external surfaces for the one or more teeth of the dental anatomy, as determined using the OCT data; and the external surfaces for the one or more teeth of the dental anatomy, as determined using the CBCT data, wherein a weighting for the external surfaces, as determined using the OCT data, is greater than a weighting for the external surfaces, as determined using the CBCT data.

9. The system of claim 1, wherein the generating of the 3D model of the dental anatomy comprises identifying gingiva of the dental anatomy based on the OCT data.

10. The system of claim 1, wherein the generating of the 3D model of the dental anatomy comprises identifying, based on the CBCT data, at least one of:
a root for at least one tooth of the dental anatomy;
a pulp cavity for at least one tooth of the dental anatomy; or
a jawbone of the dental anatomy.

11. The system of claim 1, wherein the actions further comprise:
receiving color image data of the dental anatomy; and
mapping the color image data onto external surfaces of the 3D model of the dental anatomy.

12. The system of claim 1, wherein the actions further comprise generating a visualization of the 3D model of the dental anatomy, and displaying the visualization to a user.

13. A dental imaging system comprising:
at least one processor; and
data storage, on which is stored instructions that, when executed by the at least one processor, cause the system to perform actions comprising:
receiving Optical Coherence Tomography (OCT) data of the dental anatomy;
generating, based on the OCT data, a three-dimensional (3D) model of the dental anatomy, wherein the generating of the 3D model of the dental anatomy comprises identifying one or more estimated pathology volumes within the dental anatomy, based on the OCT data; and
for each of the one or more estimated pathology volumes, predicting whether an actual pathology volume extends deeper within a corresponding tooth of the dental anatomy than the estimated pathology volume in question.

14. The system of claim 13, further comprising displaying an indication to a user that Cone Beam Computed Tomography (CBCT) imaging of the dental anatomy should be carried out.

15. The system of claim 14, wherein the indication specifies one or more teeth having one or more pathology volumes that are each predicted to extend deeper within a corresponding tooth of the dental anatomy than the corresponding estimated pathology volume.

16. The system of claim 13, further comprising performing Cone Beam Computed Tomography (CBCT) imaging of the dental anatomy.

17. The system of claim 13, wherein at least some of the one or more pathology volumes each correspond to a caries, a fracture, or a resorption.

18. A method of imaging a dental anatomy comprising:
receiving Optical Coherence Tomography (OCT) data of the dental anatomy;
receiving Cone Beam Computed Tomography (CBCT) data of the dental anatomy; and
generating a three-dimensional (3D) model of the dental anatomy based on the CBCT and OCT data,
wherein the generating of the 3D model of the dental anatomy comprises identifying one or more pathology volumes within the dental anatomy, based on the CBCT and OCT data.

19. The method of claim 18, wherein identifying one or more pathology volumes within the dental anatomy comprises:
determining, based on the OCT data, one or more estimated pathology volumes; and
determining, based on the CBCT data, and the one or more estimated pathology volumes, the one or more pathology volumes within the dental anatomy.

20. The method of claim 18, wherein generating the three-dimensional (3D) model of the dental anatomy comprises:
determining, based on the OCT data, external surfaces for one or more teeth of the dental anatomy;
determining, based on the CBCT data, external surfaces for the one or more teeth of the dental anatomy; and
registering the CBCT data relative to the OCT data, based on the external surfaces for the one or more teeth, as determined using the OCT and CBCT data.

21. The method of claim 18, wherein generating the three-dimensional (3D) model of the dental anatomy comprises:
determining, based on the OCT data, external surfaces for one or more teeth of the dental anatomy; and
determining, based on the CBCT data, external surfaces for the one or more teeth of the dental anatomy; and
wherein the method further comprises: identifying at least one of an artifact region, or a missing region in the external surfaces for the one or more teeth determined using the OCT data; and
correcting the at least one of the artifact region, or the missing region, based on a corresponding at least one region of the external surface for the one or more teeth determined using the CBCT data.

22. A method of imaging a dental anatomy comprising:
receiving Optical Coherence Tomography (OCT) data of the dental anatomy;
generating, based on the OCT data, a three-dimensional (3D) model of the dental anatomy, wherein the generating of the 3D model of the dental anatomy comprises identifying one or more estimated pathology volumes within the dental anatomy, based on the OCT data; and
for each of the one or more estimated pathology volumes, predicting whether an actual pathology volume extends deeper within a corresponding tooth of the dental anatomy than the estimated pathology volume in question.

23. A non-transitory computer readable storage medium, on which is stored instructions that, when executed by at least one processor of a dental imaging system, cause the system to perform actions comprising:
receiving Optical Coherence Tomography (OCT) data of the dental anatomy;
receiving Cone Beam Computed Tomography (CBCT) data of the dental anatomy; and
generating a three-dimensional (3D) model of the dental anatomy based on the CBCT and OCT data,
wherein the generating of the 3D model of the dental anatomy comprises identifying one or more pathology volumes within the dental anatomy, based on the CBCT and OCT data.

24. The non-transitory computer readable storage medium of claim 23, wherein identifying one or more pathology volumes within the dental anatomy comprises:
determining, based on the OCT data, one or more estimated pathology volumes; and
determining, based on the CBCT data, and the one or more estimated pathology volumes, the one or more pathology volumes within the dental anatomy.

25. The non-transitory computer readable storage medium of claim 23, wherein generating the three dimensional (3D) model of the dental anatomy comprises:
determining, based on the OCT data, external surfaces for one or more teeth of the dental anatomy;
determining, based on the CBCT data, external surfaces for the one or more teeth of the dental anatomy; and
registering the CBCT data relative to the OCT data, based on the external surfaces for the one or more teeth, as determined using the OCT and CBCT data.

26. The non-transitory computer readable storage medium of claim 23, wherein generating the three-dimensional (3D) model of the dental anatomy comprises:
determining, based on the OCT data, external surfaces for one or more teeth of the dental anatomy; and
determining, based on the CBCT data, external surfaces for the one or more teeth of the dental anatomy; and
wherein the method further comprises: identifying at least one of an artifact region, or a missing region in the external surfaces for the one or more teeth determined using the OCT data; and
correcting the at least one of the artifact region, or the missing region, based on a corresponding at least one region of the external surface for the one or more teeth determined using the CBCT data.

27. A non-transitory computer readable storage medium, on which is stored instructions that, when executed by at least one processor of a dental imaging system, cause the system to perform actions comprising:
receiving Optical Coherence Tomography (OCT) data of the dental anatomy;
generating, based on the OCT data, a three-dimensional (3D) model of the dental anatomy, wherein the generating of the 3D model of the dental anatomy comprises identifying one or more estimated pathology volumes within the dental anatomy, based on the OCT data; and
for each of the one or more estimated pathology volumes, predicting whether an actual pathology volume extends deeper within a corresponding tooth of the dental anatomy than the estimated pathology volume in question.
